(19) United States
(12) Patent Application Publication
(10) Pub. No.: US 2022/0054355 A1
(43) Pub. Date: Feb. 24, 2022

(54) BODY CARE MOTION TRACKING DEVICE AND BODY CARE MANAGEMENT METHOD USING SAME

(71) Applicant: GIRIM INC, Daejeon (KR)

(72) Inventor: Jeong Eun KIM, Daejeon (KR)

(73) Assignee: GIRIM INC, Daejeon (KR)

(21) Appl. No.: 17/299,541

(22) PCT Filed: Nov. 13, 2019
(86) PCT No.: PCT/KR2019/015432

§ 371 Date: Jun. 3, 2021

(30) Foreign Application Priority Data
Dec. 4, 2018 (KR) 10-2018-0154538

(51) Int. Cl. A61H 99/00

(52) U.S. Cl. CPC A61H 99/00; A61H 2201/5043; A61H 2201/5084; G16H 70/20; A61H 2201/5092; A61H 2230/62; A61H 2201/0153

(57) ABSTRACT

Proposed are a body care motion tracking device and a body care management method using the same. More specifically, proposed are a body care motion tracking device and a body care management method using the same, which are capable of: tracking body care, which is beauty management performed by manual-manipulation, through a tracking device and storing the body care having been tracked as data; and visualizing stored data and displaying the data on a display device, thereby performing user-customized manual-manipulation management on the basis of the same.

### TECHNICAL FIELD

The present disclosure relates to a body care motion tracking device and a body care management method using the same. More specifically, the present disclosure relates to a body care motion tracking device and a body care management method using the same, which are capable of: tracking body care, which is beauty management performed by manual-manipulation, through a tracking device and storing the body care having been tracked as data; and visualizing stored data and displaying the data on a display device, thereby performing user-customized manual-manipulation management on the basis of the same.

### BACKGROUND ART

With the recent development of control technology, in addition to a relatively simple massage motion such as a tapping motion, complex massage motions such as acupressure motion, kneading motion, and combinations thereof have become possible. In addition, by performing such massage operations according to a preset program using a device or the like, an effect comparable to a massage performed by an experienced masseur may be obtained. A technology related to a massage device that operates massage motions according to a preset program has been described in Japanese Patent Publication No. Hei 6-327739 (a chair-type massage device with an automatic course function, hereinafter referred to as conventional art). The conventional art includes a repetition instruction unit configured to instruct repetition of the desired massage operation during execution of a massage course, and a control unit configured to re-execute the massage operation instructed by the repetition instruction unit at that point of time. According to such a configuration, it is possible to quickly respond to a request of a user who is receiving a massage course and to improve the user's satisfaction with the massage course.

However, the massage device as described above has problems in that the device provides a massage that is less refreshing than a massage that proceeds directly by manual-manipulation, is not capable of performing the massage according to the body shape and condition of each individual, and proceeds with the massage in an inflexible way, thereby causing some people to feel pain.

### DOCUMENTS OF RELATED ART

### Patent Document

Japanese Patent Publication No. Hei 6-327739 (a chair massage device with an automatic course function)

### DISCLOSURE

### Technical Problem

Accordingly, the present disclosure has been made keeping in mind the above problems occurring in the related art and is intended to solve a conventional problem of a massage that proceeds without considering a body shape and condition of a user when the user receives body care performed by an operator actually without relying on a device or the like.

In addition, the present disclosure is intended to solve a problem of not proceeding with the body care in various parts of the body and of proceeding with only almost the same parts when proceeding with the body care again later as a history has not been stored after proceeding with the body care.

### Technical Solution

In order to accomplish the above objective, the present disclosure provides a body care motion tracking device configured to perform body care to a user using a hand tool 220, the device including: a hand tracker 210 configured to track movement of the hand tool 220; a database 100 configured to store information from the hand tracker 210; a controller 200 configured to visualize the information; and a display device 300 configured to output the visualized information.

The hand tool 220 may include a Kinect sensor and an infrared stereo camera, and the hand tracker 210 may include a motion detection sensor.

The body care may be performed in a bed, and the bed may include: a posture measurement unit and a body shape measurement unit configured to measure weight and a body shape of the user, respectively.

There may be a body care management method using a body care motion tracking device, the method being configured to proceed with body care performed to a user using a hand tool 220 and comprising: measuring a posture and a body shape of the user lying on a bed; massaging the user lying on the bed using the hand tool 220; tracking movement of the hand tool 220 by a hand tracker 210; storing the measured posture and body shape and the movement of the hand tool 220 in a database; visualizing stored data; and displaying visualized data.

The hand tool 220 may preset a movement path thereof in a position table thereof.

### Advantageous Effects

As described above, in the present disclosure, even when body care, which is performed directly by manual-manipulation, proceeds, the current status of the body care performed through a hand tool and a hand tracker can be stored in a database and visualized to be confirmed.

In addition, by directly measuring the user's body and storing the user's body information, it is possible to determine the user's body shape, and body care can be performed accordingly.

In addition, it is possible to accumulate and use data by body types of various people transmitted from sensors provided in the hand tool.

### DESCRIPTION OF DRAWINGS

FIG. 1 is a view showing an overall structure according to an embodiment of the present disclosure.

FIG. 2 is a view showing appearance of a device being used according to the embodiment of the present disclosure.

FIG. 3 is a view showing appearance of the device being applied according to the embodiment of the present disclosure.

FIG. 4 is a view showing a controller according to the embodiment of the present disclosure.

FIG. 5 is a diagram showing communication of each device according to the embodiment of the present disclosure.

### MODE FOR INVENTION

Advantages and features of the embodiments of the present disclosure and a method of achieving the same will become apparent with reference to the embodiments described later in detail together with the accompanying drawings. However, the present disclosure is not limited to the embodiments disclosed below and may be implemented in a variety of different forms. Here, these embodiments make the disclosure of the present disclosure complete and are provided to completely inform the scope of the invention to those who have common knowledge in the technical field to which the present disclosure pertains, and the present disclosure is only defined by the scope of the claims. In addition, the same reference numerals refer to the same elements throughout the specification.

In describing the embodiments of the present disclosure, when it is determined that a detailed description of a known function or configuration may unnecessarily obfuscate the subject matter of the present disclosure, a detailed description thereof will be omitted. In addition, terms to be described later are terms defined in consideration of functions in an embodiment of the present disclosure, which may vary according to the intention or custom of users or operators. Therefore, the definition of such terms should be made based on the contents throughout the present specification.

The present disclosure is a device that is configured to track, store, and visualize operation data of body care performed by manual-manipulation, the device being able to analyze and visualize the contents of manual-manipulation management later on the basis of the manual-manipulation management performance data and being further able to proceed with user-customized manual-manipulation management.

Hereinafter, an embodiment of the present disclosure will be described in detail with reference to the accompanying drawings.

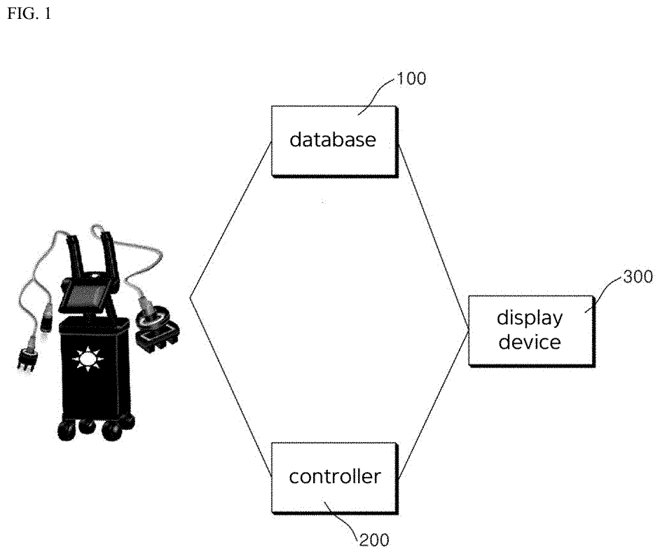

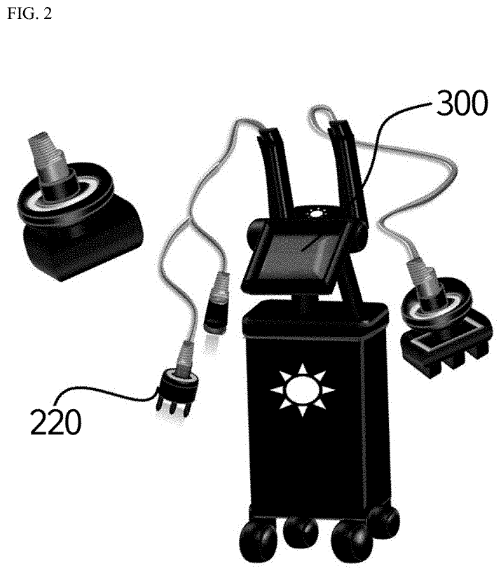

FIG. 1 is a view showing an overall structure according to an embodiment of the present disclosure, and FIG. 2 is a view showing appearance of the device being used according to the embodiment of the present disclosure.

With reference to FIG. 1, a body care motion tracking device according to an embodiment of the present disclosure is largely associated with a user, an operator, and a device, wherein the device includes a database 100, a controller 200, and a display device 300.

The user is a person receiving body care and lies on a bed, thereby receiving the proceeding body care. In this case, the bed further includes a posture measurement unit and a body shape measurement unit configured to measure the weight and body shape of the user, respectively. This will be described later.

The operator is a person performing the body care to the user and proceeds with the body care using a hand tool 220 as shown in FIG. 2. In this case, the hand tool 220 may be a tool used in a general massage shop, and any device may be used as long as the device includes a sensor capable of tracking movement.

In addition, the body care motion tracking device further includes a hand tracker 210 configured to track the movement of the hand tool 220.

In addition, the controller 200 is configured to control the motion of the hand tool 220 and the hand tracker 210 and to acquire and visualize data, and the database 100 is configured to store motion data and customer-related user data.

That is, as described above, the controller 200 controls and visualizes the motion of the hand tool 220 and the hand tracker 210, and the database 100 stores the motion data of the hand tool 220 and the data of the hand tracker 210 and further stores data from the posture measurement unit and the body shape measurement unit.

Visualized information from the controller 200 is output through the display device 300. In this case, the display device 300 may be any one of devices such as a user's terminal, a PC, and the like, as long as the device is able to display the visualized information.

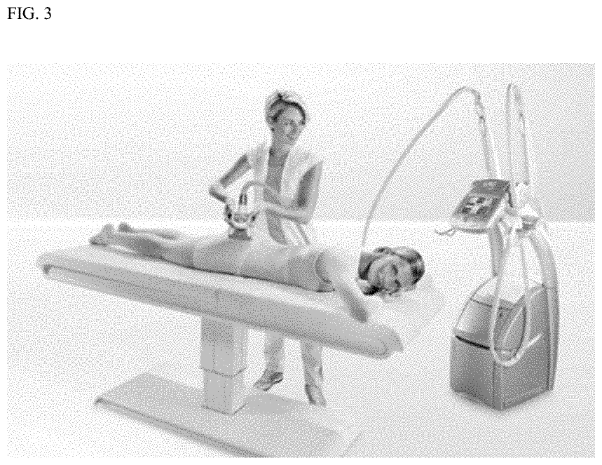

FIG. 3 is a view showing appearance of the device being applied according to the embodiment of the present disclosure. As shown in FIG. 3, the operator proceeds with the care on the user's body using the hand tool. At this time, the bed on which the user is lying down monitors the change in weight by measuring the weight of the user receiving the body care or measures the posture of the spine of the user lying on the bed, and the like.

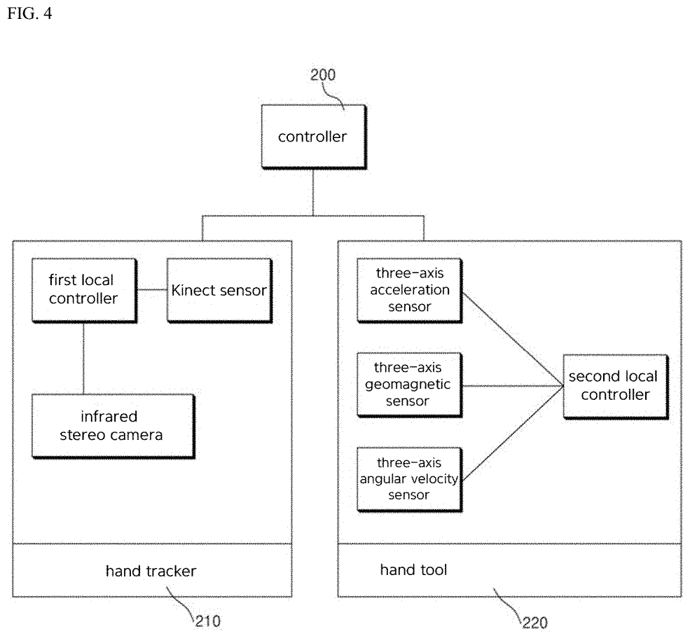

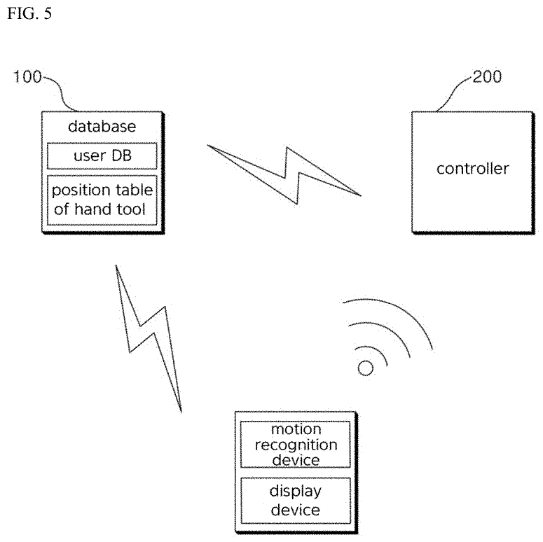

FIG. 4 is a view showing a controller according to the embodiment of the present disclosure, and FIG. 5 is a diagram showing communication of each device according to the embodiment of the present disclosure. With reference to FIGS. 4 to 5, the body care motion tracking device according to the embodiment of the present disclosure and the body care management method using the same will be described in more detail.

As described above, the controller 200 is configured to control the motion of the hand tool 220 and the hand tracker 210. The hand tool 220 is equipment configured to be directly used in a body care procedure, and the hand tracker 210 is configured to track the movement of the operator's hand holding the hand tool 220. In this case, in order to track the movement of the operator's hand, a sensor or the like may be further provided on the operator's hand.

The controller 200 is configured to control the motion of the hand tool 220 and the hand tracker 210 and acquire data, wherein the hand tracker 210 includes a first local controller, and the hand tool 220 includes a second local controller. The first local controller is a controller configured to control the hand tracker 210, is configured to obtain tracking information from the hand tracker 210 and to calculate the movement path information of the operator's hand, and includes a Kinect sensor and an infrared stereo camera.

The Kinect sensor may sense a motion by detecting a joint of the operator holding the hand tool 220, and the infrared stereo camera may acquire motion information by photographing the operator's hand. At this time, the information acquired from the Kinect sensor and the stereo camera is transmitted to the controller 200 through the first local controller.

The hand tool 220 includes the second local controller, wherein the second local controller is a controller configured to control the hand tool 220, is configured to obtain motion information from the hand tool 220 and to calculate the movement path information of the hand tool 220, and includes a three-axis acceleration sensor, a three-axis geomagnetic sensor, and a three-axis angular velocity sensor. The three-axis acceleration sensor, the three-axis geomagnetic sensor, and the three-axis angular velocity sensor are configured to measure the direction, speed, and angle of the hand tool 220 of being moved, the hand tool 220 itself is a sensor capable of detecting motion, and the value sensed by the sensor is transmitted to the controller 200 through the second local controller. Here, the three-axis acceleration sensor, the three-axis geomagnetic sensor, and the three-axis angular velocity sensor are known technologies, and detailed descriptions thereof will be omitted.

Meanwhile, the movement path of the hand tool 220 may be estimated by being compared with data of a preset position table of the hand tool. Depending on a type of the body care the user is to receive, a path value in which the hand tool 220 should basically move may be set, and the path value may be stored in advance in the position table of the hand tool.

In addition, the device may further include a motion data calculator (not shown). The motion data calculator may finally calculate the movement path of the hand tool 220 while the body care procedure is being performed. For example, the motion information of only the hand tool 220 held in the operator's hand may include motions that are not involved in the procedure (personal actions of the operator and the like), so a step of finally calculating the movement path of the hand tool 220 is further performed. That is, the body care motion tracking device according to the embodiment of the present disclosure may correct motion information of the hand tool 220 using the information of the hand tracker 210.

In addition, the motion data calculator calculates the movement path of the hand tool 220 and finally stores the same in the database 100 to be described later. At this time, as described above, the information on the motion not involved in the procedure is stored like noise together in the data of the movement path.

Such noise is likely to act as an error of the data in accuracy analysis or history management performed in the future for the procedure received by the user. Therefore, for the motions not involved in the procedure, the corresponding data may be classified and stored as noise in the database 100.

Specifically, in order to separate the motion data not involved in the procedure from the motion data related to the procedure, a learning method through machine learning may be used for the motion data (a form including acceleration sensor data and the like).

In one embodiment, the motion data may be trained into a plurality of layers using a convolutional neural network (CNN) method among deep neural network methods of classifying such data. In particular, the operator's motion pattern or the user's motion pattern always forms a data pattern in a certain form for the same procedure. Therefore, by being paid attention to this point, the noise of the data pattern (non-procedural movement) may be distinguished using Supported Vector Machine (SVM).

In particular, in the case of the SVM, the data acquired through the acceleration sensor is vectorized, thereby being trained about the vector. Accordingly, the pattern vector of the acceleration sensor value generated during the operation related to the procedure and the pattern vector of the acceleration sensor value generated in the non-procedural action are effectively distinguished. As a result, the noise related to the non-procedures may be effectively removed from the database.

Looking at a method of performing body care performed using the hand tool 220 to the user, first, a step of measuring the posture and body shape of the user lying on the bed is performed.

In addition, it is possible to accumulate and use data for each body type of various people transmitted from the sensors provided in the hand tool. That is, the hand tool may further include sensors and may accumulate and utilize data for each body type of several people transmitted from the sensors.

The user lying on the bed provides the information thereof and is measured for changes in posture during receiving the body care along with the weight and body shape. At this time, the measured data is transmitted to the database 100.

A step of massaging the user lying on the bed is performed using the hand tool 220, and a step of tracking the movement of the hand tool 220 is performed by the hand tracker 210. The operator proceeds with the user's body care using the hand tool 220 equipped with the hand tracker 210. In this case, the information on the movement path of the hand is saved by the hand tool 220 and the hand tracker 210. The information stored in this case is stored in the database 100.

A step of visualizing the stored data and a step of displaying the visualized data are performed. The information stored in the database 100 is stored so that the user may check the contents of his/her manual-manipulation management later, and the body care program received by the user may be checked by eye through the visualized information.

That is, the database 100, the controller 200, and the display device 300 communicate with each other to exchange information, and the database 100 stores personal information such as user information, for example, the body shape, the weight, and the movement during the body care, and also stores the position table of the hand tool 220. The information stored in the database 100 is visualized by communicating with the controller 200, and both the information visualized by the controller 200 and the information stored in the database 100 are output to the display device 300, thereby being allowed to be checked.

The display device 300 is configured to show the final movement path of the hand tool 220 calculated by the motion data calculator in a two-dimensional or three-dimensional graphic. The user and the operator may check the movement path of the hand tool 220 shown through the display with eyes, thereby checking the type of body care treatment received by the user, the accuracy of the treatment, and the effect of the treatment, and the like and also checking the body care history information, body, body shape change information, and the like of the user.

In this case, wireless Internet may be used to communicate with each other, but the present disclosure is not limited thereto, and any device may be used as long as the device is able to communicate.

In addition, the body care motion tracking device according to one embodiment of the present disclosure is used by the user lying down on the bed according to the embodiment but is also possible to be used by the user standing up. In addition, the device may also be used by the user sitting down on the bed according to another embodiment.

In the above description, various embodiments of the present disclosure have been presented and described, but the present disclosure is not necessarily limited thereto, and those of ordinary skill in the art to which the present disclosure pertains will readily appreciate that various substitutions, modifications, and changes are possible within the scope of the technical spirit of the present disclosure.

1. A body care motion tracking device configured to perform body care to a user using a hand tool (220), the device comprising:
a hand tracker (210) configured to track movement of the hand tool 220;
a database (100) configured to store information from the hand tracker (210);
a controller (200) configured to visualize the information; and
a display device (300) configured to output the visualized information.

2. The device of claim 1, wherein the hand tool (220) comprises a Kinect sensor and an infrared stereo camera, and
the hand tracker (210) comprises a motion detection sensor.

3. The device of claim 1, wherein the body care is performed in a bed, and the bed comprises:
a posture measurement unit and a body shape measurement unit configured to measure weight and a body shape of the user, respectively.

4. A body care management method using a body care motion tracking device, the method being configured to proceed with body care performed to a user using a hand tool (220) and comprising:
measuring a posture and a body shape of the user lying on a bed;
massaging the user lying on the bed using the hand tool (220);
tracking movement of the hand tool (220) by a hand tracker (210);
storing the measured posture and body shape and the movement of the hand tool (220) in a database;
visualizing stored data; and
displaying visualized data.

5. The method of claim 4, wherein the hand tool (220) presets a movement path thereof in a position table thereof.
